(19) United States
(12) Patent Application Publication
(10) Pub. No.: US 2005/0170498 A1
(43) Pub. Date: Aug. 4, 2005

(54) Multiwell plate and method for making multiwell plate using a low cytotoxicity photocurable adhesive

(76) Inventors: Paula J. Dolley, Corning, NY (US); Mark A. Lewis, Corning, NY (US); Gregory R. Martin, Acton, ME (US); Kevin R. McCarthy, Horsehead, NY (US); Paul J. Shustack, Elmira, NY (US); Kimberly S. Wayman, Gillet, PA (US); Michael J. Winningham, Big Flats, NY (US)

Correspondence Address: CORNING INCORPORATED, SP-TI-3-1, CORNING, NY, 14831, US

(21) Appl. No.: 11/046,427

(22) Filed: Jan. 28, 2005

Related U.S. Application Data
(60) Provisional application No. 60/540,918, filed on Jan. 30, 2004.

(51) Int. Cl. C12M 1/34

(52) U.S. Cl. 435/288.4; 435/305.2; 422/102

(57) ABSTRACT

A multiwell plate is described herein that can be used in cell-based applications and is made from a plastic upper plate which forms the sidewalls of one or more wells and a glass lower plate which forms the bottom walls of the wells. The plastic upper plate and glass lower plate are attached and bound to one another by a cationically photocured adhesive. A preferred cationically photcured adhesive includes: (1) one or more photocationally polymerizable epoxy and/or oxetane functional resins; (2) a low fluorescing cationic photoinitiator; and (3) a low fluorescing photosensitizer if the cationic photoinitiator does not have an adequate absorption at a wavelength >280 nm to initiate cure. Also described herein is a method for making such multiwell plates.

This application claims the benefit of U.S. Provisional Application Ser. No. 60/540,918 filed on Jan. 30, 2004 and entitled “Multiwell Plate and Method for Making Multiwell Plate Using a Low Cytotoxicity Photocurable Adhesive” which is incorporated by reference herein.

### BACKGROUND OF THE INVENTION

1. Field of the Invention

The present invention relates in general to the biotechnology field and, in particular, to a multiwell plate made from a plastic upper plate and a glass bottom plate that are bonded to one another with a low cytotoxicity photocurable adhesive.

2. Description of Related Art

Today biochemical studies associated with growing cells and other cell-based assays are carried out on a large scale in both industry and academia, so it is desirable to have an apparatus that allows these studies to be performed in a convenient and inexpensive fashion. Because they are relatively easy to handle and low in cost, multiwell plates are often used for such studies.

One type of multiwell plate used to perform cell-based assays is made from a plastic frame which forms the sidewalls of a matrix of wells and a glass plate which forms the bottoms of the wells. The glass plate which is transparent can be made extremely flat and has a surface that lends itself very well to various surface treatments. And, the plastic frame can be easily fabricated by injection molding plastic. To bond the plastic frame to the glass plate, an adhesive is necessary. Since glass is transparent, it is desirable to use a photocurable adhesive for this purpose. However, not all photocurable adhesives for bonding glass to plastic work in cell-based applications. This is largely true for two reasons. First, if the multiwell plate is to be used for cell-based assays, then the adhesive must be cell compatible or non-cytotoxic. Because, no matter how the multiwell plate is assembled, there will always be some adhesive around the perimeter of the bottom of the well which is going to contact the cell culture solution in the wells. And, if the adhesive is cytotoxic then it will adversely affect the cell growth. Secondly, many cell-based assays depend on fluorescence techniques to analyze the cells. As such, the cured adhesive must not fluoresce appreciably at the excitation wavelengths or it can interfere with the study.

Most of the commercially available ultraviolet (UV) curable adhesives fail either the cytotoxity or fluorescence requirements or simply do not possess enough adhesion to the glass plate or plastic frame to remain adequately bonded to one another. For instance, the glass bottom multiwell plates currently on the market such as Greiner's SensoPlate® and BD Bioscience's BD Falcon™ Glass-Bottom Imaging Plate are manufactured with acrylic adhesives that have problems with odor, volatiles, extractables and cytotoxicity. These issues are common issues with the use of acrylate and methacrylate based adhesives. Adhesives formulated with acrylates tend to have significant volatile components (typically several percent of the formulation) in the uncured state. These volatiles can be avoided by using higher molecular weight oligomers, but the viscosity becomes very high and then the problematical lower molecular weight monomers (volatile) are generally required to reduce the viscosity. The acrylic adhesives also suffer from incomplete cure at the surface when oxygen is present and from cure termination when the ultraviolet light used to cure them is turned off. Both of these effects lead to incomplete cure which can polute the multiwell plate surface through outgasing and extraction when the cell culture solution is in the wells. For Greiner and BD Bioscience this means that their plates namely the SensoPlate® and the BD Falcon™ Glass-Bottom Imaging Plate can not be used for cell-based applications.

Table 1 is provided below which shows the test results that were obtained when glass bottom multiwell plates made with different commercially available adhesives where tested to determine if they had acceptable properties of adhesion, fluorescence, cytotoxicity and odor.

TABLE #1

| Name of | | Type of | |
|---|---|---|---|
| Adhesive | Manufacturer | Adhesive | Test Results |
| VTC02 | Summers | UV Cure | Failed 72 hour adhesion |
| NOA-63 | Norland | UV Cure | Failed 72 hour adhesion |
| 3494 | Loctite | UV Cure | Adhesive took up color |
| | | | from cell media |
| 3336 | Loctite | UV Cure/ | Failed 72 hour adhesion |
| | | Epoxy | |
| 140-M | Dymax | UV Cure | Failed autofluorescence |
| 1-20387 | Dymax | UV Cure | Failed autofluorescence |
| 1-20395 | Dymax | UV Cure | Failed cytotoxicity |
| UV10- | Masterbond | UV Cure | Failed autofluorescence |
| medical | | | and cytotoxicity |
| OGRFI-146 | Ablestik | UV Cure | Failed cytotoxicity |
| 425 | | UV Cure | Failed autofluorescence |
| J91 | Summers | UV Cure | Failed autofluorescence |
| UV74 | Summers | UV Cure | Failed autofluorescence |
| | | | and Foul odor |
| NOA-60 | Norland | UV Cure | Failed autofluorescence |
| NOA-61 | Norland | UV Cure | Failed autofluorescence |
| NOA-65 | Norland | UV Cure | Failed autofluorescence |
| NOA-68 | Norland | UV Cure | Failed autofluorescence |
| NOA-72 | Norland | UV Cure | Failed cytotoxicity |
| NOA-76 | Norland | UV Cure | Failed cytotoxicity |
| UVA-4103 | Star Technology | UV Cure | Failed 72 hour adhesion |
| UVE-4101 | Star Technology | UV Cure | Failed autofluorescence |
| 4L53 | Permabond | UV Cure | Failed autofluorescence |
| 4L25 | Permabond | UV Cure | Failed autofluorescence |
| XSD 1422 | Crosslink | UV Cure | Failed autofluorescence |
| | Technology | | |
| Abelux | Ablestik | UV Cure | Failed 72 hour adhesion |
| A4083 | | | and cytotoxicity |
| Abelux | Ablestik | UV Cure | Failed autofluorescence |
| A4088 | | | |
| ST-3500 | Star Technology | UV Cure | Failed cytotoxicity |
| L-25-2 | Holdtite | UV Cure | Failed 72 hour adhesion |
| ELC 4481 | Electrolite | UV Cure | Failed autofluorescence |
| Dymax 141M | Dymax | UV Cure | Failed autofluorescence |

As can be seen, the glass bottom multiwell plates made with these commercially available adhesives should not be used for cell-based applications. Accordingly, there is a need for an adhesive that can then be used to make a glass bottom multiwell plate which can be used to perform cell-based assays. This need and other needs are satisfied by the cationically photocurable adhesive of the present invention.

### BRIEF DESCRIPTION OF THE INVENTION

The present invention includes a multiwell plate that can be used in cell-based applications and is made from a plastic upper plate which forms the sidewalls of one or more wells and a glass lower plate which forms the bottom walls of the wells. The plastic upper plate and glass lower plate are attached and bound to one another by a cationically photocured adhesive. A preferred cationically photcured adhesive includes: (1) one or more photocationally polymerizable epoxy and/or oxetane functional resins; (2) a low fluorescing cationic photoinitiator; and (3) a low fluorescing photosensitizer if the cationic photoinitiator does not have an adequate absorption at a wavelength >280 nm to initiate cure. The present invention also includes a method for making such multiwell plates.

### BRIEF DESCRIPTION OF THE DRAWINGS

A more complete understanding of the present invention may be had by reference to the following detailed description when taken in conjunction with the accompanying drawings wherein:

FIG. 1 is a perspective view of a multiwell plate in accordance with the present invention;

FIG. 2 is a cut-away partial perspective view of the multiwell plate shown in FIG. 1;

FIG. 3 is a cross-sectional side view of the multiwell plate shown in FIG. 1;

FIG. 4 is a micrograph showing the cell growth on a 384 well glass bottom microplate that was assembled using a cationically photocured adhesive (Loctite 3337) in accordance with one embodiment of the present invention;

FIG. 5 is a graph that illustrates the fluorescence curves at an 300 nm excitation wavelength for the Loctite 3337 adhesive, Loctite 3340 adhesive, Norland NOA63+2 1/2 % Silquest A-174 adhesive and Example Adhesive #s 1, 3 and 4;

FIG. 6 is a graph that illustrates the fluorescence curves at an 300 nm excitation wavelength for the Loctite 3337 adhesive, Norland NOA63+2%% Silquest A-174 adhesive and Example Adhesive #s 1, 5, 6, 7 and 8;

FIG. 7 is a graph that illustrates the fluorescence curves at an 300 nm excitation wavelength for the Loctite 3337 adhesive, Loctite 3340 adhesive, Norland NOA63 adhesive and Example Adhesive #9;

FIG. 8 is a graph that illustrates the fluorescence curves at an 300 nm excitation wavelength for the Loctite 3340 adhesive, Norland NOA63 adhesive and Example Adhesive #s 10 and 11;

FIG. 9 is a graph that illustrates the fluorescence curves at an 365 nm excitation wavelength for the Loctite 3340 adhesive, Norland NOA63 adhesive and Example Adhesive #s 10 and 11;

FIG. 10 is a graph that illustrates the fluorescence curves at an 300 nm excitation wavelength for the Loctite 3340 adhesive, Norland NOA63 adhesive and Example Adhesive #s 12, 13 and 14;

FIG. 11 is a graph that illustrates the fluorescence curves at an 365 nm excitation wavelength for the Loctite 3340 adhesive, Norland NOA63 adhesive and Example Adhesive #s 12, 13 and 14;

FIG. 12 is a block diagram of a bonding fixture that was used to help perform a tensile adhesion test on Example Adhesive #1-14;

FIG. 13 is a graph that illustrates the tensile adhesive strengths after 72 hours in 50° C. water for the Loctite 3337 adhesive, Loctite 3340 adhesive and Example Adhesive #s 1, 2, 3 and 9;

FIG. 14 is a graph that illustrates the cytotoxity data for the Loctite 3337 adhesive and Example Adhesive # 1;

FIG. 15 is a graph that illustrates the cytotoxity data for the Loctite 3337 adhesive, Loctite 3340 adhesive and Example Adhesive #s 12 and 13; and

FIG. 16 is a flowchart illustrating the steps of a preferred method for making the multiwell plate in accordance with the present invention.

### DETAILED DESCRIPTION OF THE DRAWINGS

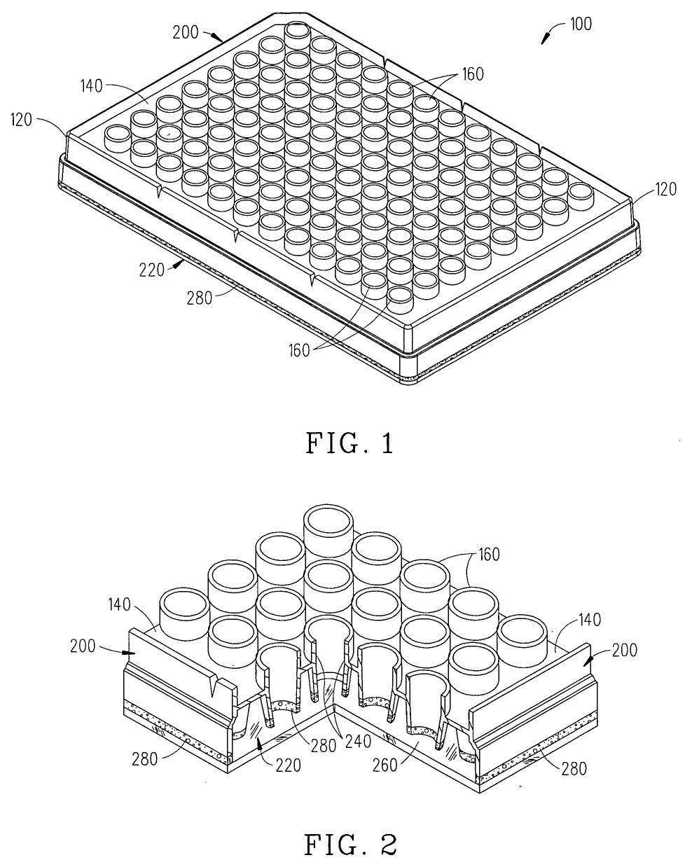

Referring to FIG. 1, there is illustrated a perspective view of an exemplary multiwell plate 100 of the present invention. The multiwell plate 100 (e.g., microplate 100) includes a peripheral skirt 120 and a top surface 140 having an array of wells 160 each of which is capable of receiving an aliquot of sample to be assayed. Preferably, the multiwell plate 100 conforms to industry standards for multiwell plates; that is to say, the multiwell plate 100 is bordered by a peripheral skirt 120, laid out with ninety-six wells 160 in an 8×12 matrix (mutually perpendicular 8 and 12 well rows). In addition, the height, length, and width of the multiwell plate 100 preferably conform to industry standards. However, the present invention can be implemented in a multiwell plate that has any number of wells and is not limited to any specific dimensions and configurations.

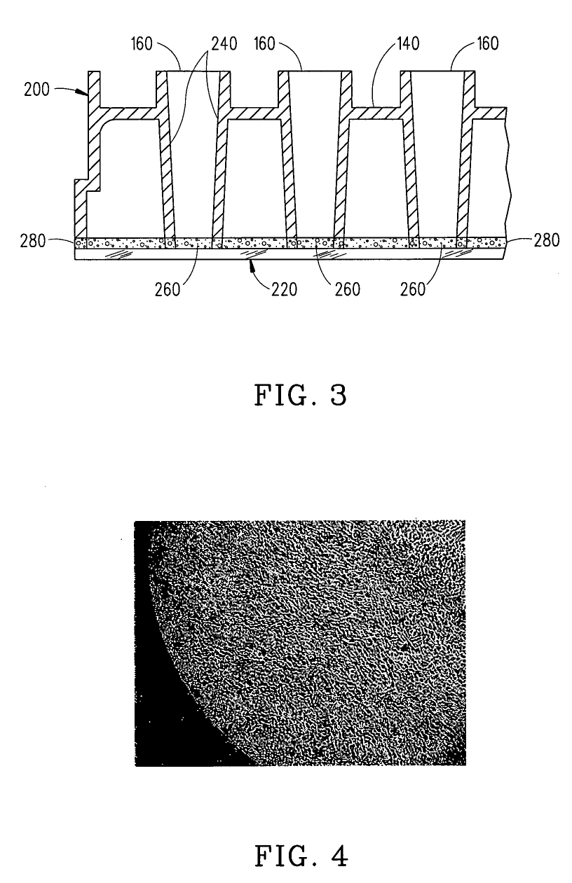

Referring to FIGS. 2 and 3, there are illustrated two cross sectional views of the multiwell plate 100 shown in FIG. 1. The multiwell plate 100 is of two-part construction including an upper plate 200 and a lower plate 220. The upper plate 200 forms the peripheral skirt 120, the top surface 140 and the sidewalls 240 of the wells 160. The lower plate 220 forms the bottom walls 260 of the wells 160. During the manufacturing process, the upper plate 200 and lower plate 220 are joined together at an interface by a cationically photocured adhesive 280. A more detailed discussion about the manufacturing process and the cationically photocured adhesive 280 is provided below after a brief discussion about the exemplary structures of the multiwell plate 100.

The upper plate 200 includes a frame that forms the sidewalls 240 of an array of open-ended sample wells 160 in addition to the peripheral skirt 120, and the top surface 140. The upper plate 200 is preferably molded from a polymeric material (e.g., polystyrene) that becomes intertwined upon heating and bonds together in a non-covalent mechanism upon cooling, thereby forming an interpenetrating polymer network. Further, the upper plate 200 need not be molded, instead the upper plate 200 can be laminated so that each layer has desired properties. For example, a top most layer may be anti-reflective, a middle layer may form the sidewalls of the wells and can be hydrophobic for meniscus control, and the bottommost layer may be a polymeric material.

The lower plate 220 is preferably made from a layer of glass material that can be purchased as a sheet from a variety of manufacturers (e.g. Corning, Inc., Erie Scientific). This sheet can then be altered to fit the dimensions of the desired size multiwell plate 100. The glass material forms a transparent bottom wall 260 for each sample well 160 and permits viewing therethrough. The transparent lower plate 220 also allows for light emissions to be measured through the bottom walls 260. As shown, the lower plate 220 is substantially flat and is sized to form the bottom walls 260 for all of the wells 160 of the upper plate 200. It should be noted that one or more chemically active coatings (not shown) can be added to a top surface of the bottom walls 260.

In the preferred embodiment, the glass is of a high optical quality and flatness such as boroaluminosilicate glass (Corning Inc. Code 1737). Optical flatness of the bottom walls 260 of the wells 160 is important particularly when the multiwell plate 100 is used for microscopic viewing of specimens and living cells within the wells 160. This flatness is also important in providing even cell distribution and limiting optical variation. For example, if the bottom wall 260 of a well 160 is domed, the cells will tend to pool in a ring around the outer portion of the bottom 260. Conversely, if the bottom wall 260 of a well 160 is bowed downwards, the cells will pool at the lowest point. Glass microscope slides are typically flat within microns to ensure an even distribution. Preferably, the bottom walls 260 of the wells 160 are formed from a glass sheet having a thickness similar to microscope slide cover slips, which are manufactured to match the optics of a particular microscope lens. Although the bottom walls 260 may be of any thickness, for microscopic viewing it is preferred that the bottom wall 260 thickness is less than or equal to 500 microns and their flatness is in the range of 0-10 microns across the diameter of the outer bottommost surface of an individual well 160.

Although the lower plate 220 as a whole is substantially flat, it may have relief features formed upon its surface such as ridges, curves, lens, raised sections, diffraction gratings, dimples, concentric circles, depressed regions, etc. Such features may be located on the lower plate 220 such that they shape or otherwise become features of the bottom walls 260 themselves, and may in turn enhance the performance of an assay, enhance or enable detection (as in the case with lenses and gratings), or serve to mechanically facilitate bonding with the upper plate 200. These relief features may be formed by any number of known methods including vacuum thermoforming, pressing, or chemical etching, laser machining, abrasive machining, embossing, or precision rolling.

Moreover, the wells 160 can be any volume or depth, but in accordance with the 96 well industry standard, the wells 160 preferably have a volume of approximately 300 ul and a depth of approximately 12 mm. Spacing between wells 160 is approximately 9 mm between center lines of rows in the x and y directions. The overall height, width, and length dimensions of the multiwell plate 100 are preferably standardized at 14 mm, 85 mm and 128 mm, respectively. Wells 160 can be made in any cross sectional shape (in plan view) including, square sidewalls 240 with flat or round bottoms, conical sidewalls 240 with flat or round bottoms, and combinations thereof.

The preferred process of manufacturing the multiwell plate 100 of the present invention includes using a cationically photocurable adhesive 280 to join the upper plate 200 and the lower plate 220. The use of the cationically photocurable adhesive 280 to bond together the upper plate 200 and the lower plate 220 of the multiwell plate 100 is a marked improvement over the traditional multiwell plate in that the multiwell plate 100 of the present invention performs well under normal cell culture conditions. In contrast, the traditional adhesives (e.g., acrylic adhesives) used to make a multiwell plate do not perform well under normal cell culture conditions because the adhesive bond that holds together the plastic upper plate and glass lower plate often degrades such that the two plates can easily separate or the contents in one well can leak into other wells (see TABLE #1). In addition, the traditional adhesives (e.g., acrylic adhesives) used to make a multiwell plate do not perform well under normal cell culture conditions because the adhesive would also fail the fluorescence, cytotoxicity and/or odor requirements.

Following are descriptions of several different cationically photocurable adhesive 280 some of which could be used to make a multiwell plate 100 in accordance with the present invention. The first cationically photocurable adhesive 280 discussed is one currently sold under the brand name of Loctite 3337. Loctite 3337 has the following composition:
Epoxy resins: 30-60 wt %
Phenol, polymer with formaldehyde, glycidyl ether: 10-30 wt %
Ethanol, 2, 2′-oxybis: 1-5 wt %
Gamma-Glycidoxypropyl trimethoxysilane: 1-5 wt %
Antimony salt: 0.1-1 wt %

In experiments, Loctite 3337 has been used to assemble 96 and 384 well glass bottom multiwell plates 100 as well as Gamma Amino Propyl Silane (GAPS) coated glass bottom multiwell plates 100 and Ta2O5 coated topas or glass bottom multiwell plates 100. These multiwell plates 100 have been used to grow cells as well if not better than Tissue Culture Treated (TCT) plates and have survived incubation of 10% FBS/media for 10 days at 37° C., as well as incubations with GPCR buffer and 5% DMSO (see FIG. 4). In addition, Loctite 3337 has been dispensed onto GAPS II slides, left uncured for 2 minutes, cured and then repackaged. After 2 months in storage the slides had no measurable change in contact angle or colloidal gold staining right up to the edge of the adhesive. One drawback with Loctite 3337 is that it forms autofluorescent species during the UV cure due to it's use of triarylsulphonium salts as the photoinitiator. This autofluorescent property can be eliminated by the use of commercially available iodonium salts (such as GE Silicone's UV9385C, Rhodia's RP-2074 and Ciba's Irgacure 250) photoinitiators that do not form fluorescent species.

The second cationically photocurable adhesive 280 discussed is one currently sold under the brand name of Loctite 3340. Loctite 3340 has the following composition:
Epoxy resin: 20-40 wt %
Cycloaliphatic epoxy resin: 10-20 wt %
Polyol: 20-30 wt %
Silica, amorphous, fumed, crystalline-free: 1-5 wt %
Carbonate: 0.5-1 wt %
Substituted silane: 1-5 wt %

Loctite 3340 and other adhesive candidates including the aforementioned Loctite 3337 have been examined by various off-line tests so as to obtain material properties information which can be used to define a desirable cationically photocurable adhesive 280. The different types of adhesive candidates tested and their associated material properties are provided below in TABLE #2.

TABLE #2

| | | | Glass | | Avg. | |
|---|---|---|---|---|---|---|
| | Post | Young's | Transition | Tensile | curl, 4 | |
| Adhesive | Condition- | Modulus | on Temp | Adhesion | specimens | |
| ID | ing Step | (MPa) | (° C.) | (MPa) | (mm) | Comments |
| *Adhesive | >16 hr, | 0.73 | −35 | 0.12 | ND | Inadequate |
| #1A | 23 C., 50% | | | | | mechanical |
| | RH | | | | | and thermal |
| | | | | | | properties |
| *Adhesive | >16 hr, | 0.81% | −42 | 0.03 | ND | Inadequate |
| #2A | 23 C., 50% | | | | | mechanical |
| | RH | | | | | and thermal |
| | | | | | | properties |
| *Adhesive | >16 hr, | 2039.04 | ND | ND | 56 | High curl, |
| #3A | 23 C., 50% | | | | | poor glass |
| | RH | | | | | adhesion |
| *Adhesive | >16 hr, | 1563.73 | 50 | ND | 62 | High curl, |
| #4A | 23 C., 50% | | | | | poor glass |
| | RH | | | | | adhesion |
| *Adhesive | >16 hr, | 282.21 | ND | ND | 83 | Poor glass |
| #5A | 23 C., 50% | | | | | adhesion |
| | RH | | | | | |
| *Adhesive | >16 hr, | 733.11 | ND | ND | 90 | Low curl, |
| #6A | 23 C., 50% | | | | | good glass |
| | RH | | | | | adhesion |
| NOA 63- | >16 hr, | 1070.82 | 41 | ND | 97 | No silane |
| 0 pph AP | 23 C., 50% | | | | | adhesion |
| | RH | | | | | promoter |
| | | | | | | added |
| NOA 63- | >16 hr, | 920.21 | 39 | 0.18 | | |
| 2.5 pph | 23 C., 50% | | | | | |
| AP | RH | | | | | |
| NOA 63- | >16 hr, | 413.8 | 35 | ND | ND | 5 pph |
| 5 pph AP | 23 C., 50% | | | | | methacryloxy- |
| | RH | | | | | propyltrimeth- |
| | | | | | | oxysilane |
| | | | | | | added |
| Loctite | 16 hr, 23 | 780.36 | ND | 0.31 | ND | |
| 3337 | C., 35-45% | | | | | |
| | RH | | | | | |
| Loctite | 96 hr, 23 | 279.01 | ND | 0.36 | ND | |
| 3337 | C., 35-45% | | | | | |
| | RH | | | | | |
| Loctite | 16 hr, 85 | 2303.93 | 42 | ND | ND | |
| 3337 | C. | | | | | |
| Loctite | 16 hr, 23 | 84.76 | ND | ND | ND | |
| 3337 | C., 65% RH | | | | | |
| Loctite | 16 hr, 23 | 2208.18 | ND | ND | ND | |
| 3337 | C., 0% RH | | | | | |
| Loctite | 16 hr, 85 | 2761.12 | ND | ND | ND | |
| 3337 | C. | | | | | |
| Loctite | 16 hr, 23 | 2716.54 | ND | ND | ND | |
| 3340 | C., 35-45% | | | | | |
| | RH | | | | | |
| Loctite | 96 hr, 23 | 2621.38 | ND | ND | ND | |
| 3340 | C., 35-45% | | | | | |
| | RH | | | | | |
| Loctite | 16 hr, 85 | 2495.61 | 110 | ND | ND | |
| 3340 | C. | | | | | |
| Loctite | 16 hr, 23 | 2459.36 | ND | High; | 101 | |
| 3340 | C., 65% RH | | | substrate | | |
| | | | | failed | | |
| Loctite | 16 hr, 23 | 2219.17 | ND | ND | ND | |
| 3340 | C., 0% RH | | | | | |
| Loctite | 16 hr, 85 | 2874.61 | ND | ND | ND | |
| 3340 | C. | | | | | |

*Table 3 illustrates the compositions of adhesives #1A-6A:

TABLE #3

| | | Adhesive Composition | | | | | |
|---|---|---|---|---|---|---|---|
| Component | Type | #1A | #2A | #3A | #4A | #5A | #6A |
| BR 3741 (wt %) | Acrylate | 52 | 52 | 0 | 0 | 0 | 0 |
| (Bomar Specialities) | Oligomer | | | | | | |
| BR 3731 (wt %) | Acrylate | 0 | 0 | 0 | 0 | 45 | 33 |
| (Bomar Specialities) | Oligomer | | | | | | |
| KWS4131 (wt %) | Acrylate | 0 | 0 | 0 | 10 | 0 | 0 |
| (Bomar Specialities) | Oligomer | | | | | | |
| Photomer 3016 (wt %) | Epoxy | 0 | 0 | 46 | 5 | 0 | 13 |
| (Cognis) | diacrylate | | | | | | |
| Ethoxylatednonyl- | Acrylate | 25 | 25 | 0 | 0 | 0 | 0 |
| phenol acrylate | monomer | | | | | | |
| PHOTOMER 4003 (wt %) | | | | | | | |
| (Cognis) | | | | | | | |
| Photomer 4028 (wt %) | Diacrylate | 0 | 0 | 0 | 82 | 0 | 0 |
| (Cognis) | monomer | | | | | | |
| Isocyanurate | Triacrylate | 0 | 0 | 0 | 0 | 39 | 0 |
| triacrylate | monomer | | | | | | |
| SR368D (wt %) | | | | | | | |
| (Sartomer Co.) | | | | | | | |
| Poly(ethylene | Acrylate | 0 | 20 | 0 | 0 | 15.75 | 0 |
| glycol) monoacrylate | monomer | | | | | | |
| (wt %) | | | | | | | |
| (Aldrish Chem. Co.) | | | | | | | |
| Isobornyl acrylate | Acrylate | 0 | 0 | 52.75 | 0 | 0 | 52.75 |
| SR506 (wt %) | monomer | | | | | | |
| (Sartomer Co.) | | | | | | | |
| TONE M-100 (wt %) | Acrylate | 20 | 0 | 0 | 0 | 20 | 0 |
| (Dow Chemical) | monomer | | | | | | |
| IRGACURE 819 (wt %) | Photo- | 1.5 | 1.5 | 1.0 | 1.5 | 0 | 1 |
| (Ciba Spec. Chem.) | initiator | | | | | | |
| IRGACURE 184 (wt %) | Photo- | 1.5 | 1.5 | 0.25 | 1.5 | 0.25 | 0.25 |
| (Ciba Spec. Chem.) | initiator | | | | | | |
| (3-acryloxypropyl)- | Adhesion | 1 | 0 | 5 | 0 | 1 | 5 |
| trimethoxysilane | promoter | | | | | | |
| (pph) | | | | | | | |
| IRGANOX 1035 (pph) | Antioxidant | 1 | 1 | 0 | 0.5 | 0 | 0 |
| (Ciba Spec. Chem.) | | | | | | | |
| pentaerythritol | Stabilizer | 0.03 | 0.03 | 0 | 0 | 0 | 0 |
| tetrakis(3- | | | | | | | |
| mercaptoproprionate) | | | | | | | |
| (pph) | | | | | | | |
| (Aldrich Chem. Co.) | | | | | | | |

To make adhesives #1A-#6A, their components were weighed using a balance and then placed into a container and and mixed until the solid components were thoroughly dissolved and the mixture appeared homogeneous. In particular, the compositions of adhesives #1A-6A were formulated such that the amounts of oligomer, monomer, and photoinitiator total 100 wt %; other additives such as the antioxidant were then added to the total mixture in units of pph. The oligomer and monomer(s) were blended together for at least one hour at 70° C. Thereafter, the photoinitiator(s), antioxidant and other additives were added, and blending continued for one hour.

All of the adhesives shown in TABLE #2 were then prepared as films that were cast on silicone release paper with the aid of a draw-down box having about a 0.005″ gap thickness. The adhesives were cured using a Fusion System's Ultraviolet (UV) curing apparatus with a 600 W/in D-bulb (50% power, 10 ft/min belt speed, nitrogen purge) to yield primary coatings 1-4 and comparative primary coatings C1-C3 in film form. These cured adhesives had thicknesses between about 0.003″ and 0.004″. The adhesive films were allowed to age (23° C., 50% relative humidity) for at least 16 hours prior to testing.

To perform the tests, the film samples were cut to a specified length and width (about 15 cm×about 1.3 cm). And, then Young's modulus, tensile strength at break, and elongation at break were measured using an Instron 4200 tensile tester. During this test, the film samples were stretched at an elongation rate of 2.5 cm/min starting from an initial jaw separation of 5.1 cm (see results in TABLE #2).

In the next test, the glass transition temperatures of the cured adhesive films were determined by determining the peak of the tan δ curves measured on a Seiko-5600 DMS in tension at a frequency of 1 Hz. Thermal and mechanical properties were tested in accordance with ASTM 82-997 (see results in TABLE #2).

In addition, the candidate adhesives in TABLE #2 were formed into rods by injecting the curable compositions into TEFLON tubing that had an inner diameter of about 0.025″. The adhesive samples were cured using a Fusion D bulb at a dose of about 2.6 J/cm² (measured over a wavelength range of 225-424 nm by a Light Bug model IL390 from International Light). After curing, the TEFLON tubing was stripped away, leaving rod samples of about 0.0225″ in diameter (after shrinkage due to cure). The cured rods were allowed to condition overnight in an environment having a controlled temperature of 23° C. and a controlled relative humidity of 50%. The cured rods were then tested to determine Young's modulus, tensile strength at break, and elongation at break using an Instron 4200 tensile tester. The adhesive films were tested at an elongation rate of 2.5 cm/min starting from an initial jaw separation of 5.1 cm (see results in TABLE #2).

The candidate adhesives in TABLE #2 also underwent a curl test. The curl test was performed as follows by first taking 3M PP2410 transparency films (8.5″×11″) and cutting them into 4″×11″ strips for casting films. The center strip was placed on clean glass plate with the center strip's bottom edge flush with the bottom end of plate and the center strip's top edge held with double stick tape. An outline of the film strip was made on the plate to enable the consistent placement of each strip. Second, the films were hand-cast using 5 mil casting box aligned with a 14″ Aluminum blade guide. Four films were cast for each formulation on the 4″×11″ strips using a small glass plate as a catch area for the excess coating. The films were then cured using a UV Fusion system. Thereafter, the cured film strips were immediately removed from the glass plate. Then the bottom, or untaped, edge of the film strip was dipped onto an ink pad and placed on clean paper. The outline of the film curl was then traced with a pen and the distance between film endpoints (in mm.) was measured (see results in TABLE #2).

As can be seen in TABLE #2, candidate adhesives #1A-6A for one reason or another would not perform well if they were used to make a glass bottom multiwell plate 100. However, the information obtained in these experiments enabled the inventors to identify the physical properties of a desirable cationically photocured adhesive 280 which can be used to make the multiwell plate 100. These physical properties are provided below:
Young's modulus: desirable >20 MPa; more desirable >200 MPa; most desirable >1500 MPa.
Tg: desirable >25 C; more desirable >35 C; most desirable >60 C.
Tensile adhesion: desirable >0.10 MPa; more desirable >0.15 MPa; most desirable >0.25 MPa.
Substrate curl: desirable >70 mm; more desirable >80 mm; most desirable >90 mm.
Extractables: Minimal.
Cytotoxicity: Minimal.
Fluorescence: Minimal at the typical excitation wavelengths (300-550 nm).
Bonding: Bond glass to the plastic well enough so that the two substrates remain bonded (no well-to-well leakage) after soaking in 50° C. water for 72 hours (for example).

As can be seen, the Loctite 3337 adhesive and especially the Loctite 3340 adhesive performed well in the aforementioned experiments. It is believed that what makes an adhesive such as Loctite 3340 suitable to make multilwell plates 100 is due to the combination of the following properties which are not necessarily listed in order of importance:
No to minimal cytotoxicity—want to minimize any interactions of adhesive with live cell lines.
High Young's modulus—want a modulus greater than typical optical fiber primary coatings. Higher Young's modulus usually correlates with higher cohesive strength. A high cohesive strength is desirable to prevent mechanical failures within the adhesive layer of the multiwell plate 100. An adhesive that behaves more like a structural adhesive is more desirable than an adhesive that behaves more like a pressure sensitive adhesive.
Glass transition temperature (Tg) above room temperature—want to avoid dramatic thermal transitions within normal operating range of adhesive. In general, it is desirable to have a Tg above room temperature.
High adhesive tensile strength—want to maximize the bond strength between the adhesive and the substrates to be bonded.
Minimize coating shrinkage—most UV coatings shrink upon curing; coatings/adhesives that have high shrinkage upon curing (going from liquid to solid) have a built up of stress at the polymer/substrate interface, which can lead to delaminations. The aforementioned off-line film curl test can be used to determine this coating shrinkage.
Cure kinetics/cure speed—want fast cure to get the desired adhesive properties during processing yet want heat minimization to prevent product warpage; also want to avoid heat post treatment which is sometimes necessary to complete curing.
Minimize volatilization of organic materials—want an adhesive and method of applying adhesive that minimizes or eliminates volatilization of components from adhesive which may condense on the pristine glass surface and affect cell growth or metabolism.
Low fluorescence background—a low fluorescence adhesive is desirable; however, if there is no glass surface contamination near the measurement site then this is likely not a functional issue (e.g., Loctite 3337 may be used in this situation).
It is also desirable that the adhesive not change the binding properties of the microplate surface for cell or biological molecule attachment.

Following is a detailed description about even more experiments that were conducted and different compositions of cationically photocurable adhesives some of which can be used to make the multiwell plate 100. After performing the following experiments it was determined that the compositions of the preferred cationically photocurable adhesive 280 had: (1) one or more photocationally polymerizable epoxy and/or oxetane functional resins; (2) a low fluorescing cationic photoinitiator; and (3) a low fluorescing photosensitizer if the cationic photoinitiator does not have an adequate absorption at a wavelength >280 nm to initiate cure. These types of cationically photocurable adhesives 208 are the most desirable for cell-based applications because the photocure can be done with low photoinitiator levels (<1%), there is no inhibition by oxygen, there is low shrinkage on cure, and they exhibit a postcure effect that enables the adhesive 280 to finish curing after exposure to the light. Each of these properties is described below in greater detail.

The low photoinitiator level is important because it lessens the amount of unreacted photoinitiator and unbound photofragments in the cured adhesive 280. Excess unreacted photoinitiator or photofragments can be extracted into the cell culture fluids and potentially become toxic to cells. A small amount of photoinitiator also lessens the absorption of the adhesive 280 in the excitation wavelength areas thus lessening the potential for fluorescence.

The lack of oxygen inhibition is important because the predominately used chemistry for photocuring, the free radical polymerization of acrylates, results in a layer of uncured or partially cured material on the surface of the adhesive where it is exposed to oxygen from the air (see discussion about the traditional acrylic adhesive in the Description of Related Art section). This surface is also where the cell culture fluids contact the adhesive. As such, the uncured or partially cured material can be extracted into the cell culture fluids and potentially become toxic to the cells. If the photocure is done under nitrogen (or in the absence of oxygen), it eliminates this surface inhibition effect. However, curing under nitrogen is expensive and adds complexity to the manufacturing process. In contrast to the traditional acrylic adhesives, the cationically photocured adhesives 208 are not sensitive to oxygen resulting in a much more complete surface cure and less potential for extractables/cell toxicity issues.

The low shrinkage during cure is important because the less shrinkage that occurs during cure results in enhanced adhesion because it reduces the interfacial stress that occurs during the cure. As such, a majority of the preferred cationically photocurable adhesives 208 are epoxy based. The epoxy functional groups cure by a ring opening homopolymerization mechanism which results in significantly less shrinkage on cure than the more typically used acrylate based free radical addition polymerization.

The postcure effect after exposure to actinic light is important because it means that the entire curing reaction does not need to be complete while the adhesive 280 sets underneath the UV light. The reaction can be initiated by a quick pass under UV light. Then the “dark cure” can continue until the polymerization is complete. This not only improves throughput due to less dwell time of the part under the UV light but also the shorter dwell time under the hot UV light lessens the potential for multiwell plate 100 warpage due to excess heat from the UV light.

As described above, the preferred cationically photocurable adhesives 208 have low fluorescence and maintain good cell compatibility and adhesive properties. It was found during the experiments that adhesives 208 containing iodonium type cationic photoinitiators tended to have much lower fluorescence than adhesives that contain sulfonium salts. This is because most iodonium salt photoinitiators have very little absorbance over 300 nm while the sulfonium salt photoinitiators have absorbance out to about 375 nm. And, two of the preferred fluorescence excitation wavelengths happen to be at 300 and 365 nm. As such, to minimize fluorescence one should formulate compositions of cationically photocurable adhesives 208 that have minimal absorption at the fluorescence excitation wavelengths of 300 and 365 nm. However, sometimes it is not sufficient to simply use only an iodonium salt as the photoinitiator. This is because some glass bottom multiwell plates 100 only transmit ˜40% of the light at 300 nm and <10% of the light at 280 nm. In other words, there is not enough light that comes through the glass plate 220 at the wavelengths for the iodonium salt photoinitiator to initiate cure. Therefore, in these cases a non- or very low fluorescing photosensitizer should be used that can absorb the available light and transfer the energy or a radical to the iodonium salt to form the acid that initiates the epoxy polymerization. In the cases where the transmissivity of the glass plate 220 is sufficient for the iodonium salt to initiate cure, the photosensitizer is optional. Likewise, it is also important to select epoxy resins that have minimal or no fluorescence at the excitation wavelengths. The preferred cationically photocurable adhesives 280 may use oxetane functional resins as either a substitute for the photocationically polymerizable epoxy, or preferrably as resins to be used in combination with the epoxies. In addition, polyols also can be added to the compositions of adhesives 208 to enhance properties like adhesion and flexibility. Moreover, epoxy functional silane coupling agents also can be added to the compositions of adhesives 208 to enhance adhesion to the glass plate 220.

In view of the foregoing, the preferred cationically photocurable adhesive 208 includes one or more cationically curable epoxy and/or oxetane functional resins. The epoxy functional group can be terminal, pendant, internal, or on a cyclic ring. The preferred epoxies are cycloaliphatic epoxies. The epoxy or oxetane functional resin itself should also have low fluorescence. The preferred cationically photocurable adhesive 208 also includes one or more low fluorescing cationic photoinitiators. The preferred type of cationic photoinitiators are iodonium salts. And, if the cationic photoinitiator does not have adequate absorbance at wavelengths >280 nm (or the combination of UV light output and glass transmissivity results in insufficient available light intensity at wavelengths where the photoinitiator can initiate cure), then the preferred cationically photocurable adhesive 208 would require a photosensitizer. The photosensitizer also should have low fluorescence. The cationic photoinitiator and photosensitizer (if necessary) should both be present at no more than 1% by weight each. The preferred photoinitiator/photosensitizer is Rhodia 2074 and Esacure KIP 150. Lastly, the preferred cationically photocurable adhesive 208 may also include a polyol to enhance the adhesion and flexibility properties.

Examples of cationic photoinitiators that can be used are, but are not limited to: Rhordorsil 2074 and 2076 from Rhodia, Sarcat CD-1012 from Sartomer, Irgacure 250 from Ciba Geigy, UV9392C and UV 9385C from GE Silicones, Deuteron 2257 from Deuteron GmbH, and Nisso CI-5102 from Nippon Soda.

Examples of photosensitizers that can be used are, but are not limited to: Darocur 1173, Darocur MBF, Irgacure 184, Irgacure 754, Irgacure 500, Irgacure 651, and Irgacure 2959 from Ciba Geigy, and Esacure KIP150 and Esacure KK from Lamberti, benzophenone and diethoxyacetophenone.

Examples of polyols that can be used are, but are not limited to: Polytetramethylene ether glycols (Terathane series from duPont), Polycaprolactone polyols (Tone series from Dow Chemical or CAPA series from Solvay), Polyether polyols and alkoxylated polyether polyols (Poly G series from Arch Chemical, Voranol series from Dow Chemical, Acclaim series from Bayer, Pluracol series from BASF, Sovernol series from Cognis Corp.), Acrylic polyols (Acryflow series from Lyondel), Polyester polyols (Stepanpol series from Stepan Co., Desmophen series from Bayer, K-Flex series from King Industries, Priplast series from Unichema, Fomrez series from Uniroyal) and Polycarbonate polyols (Revecarb series from Enichem).

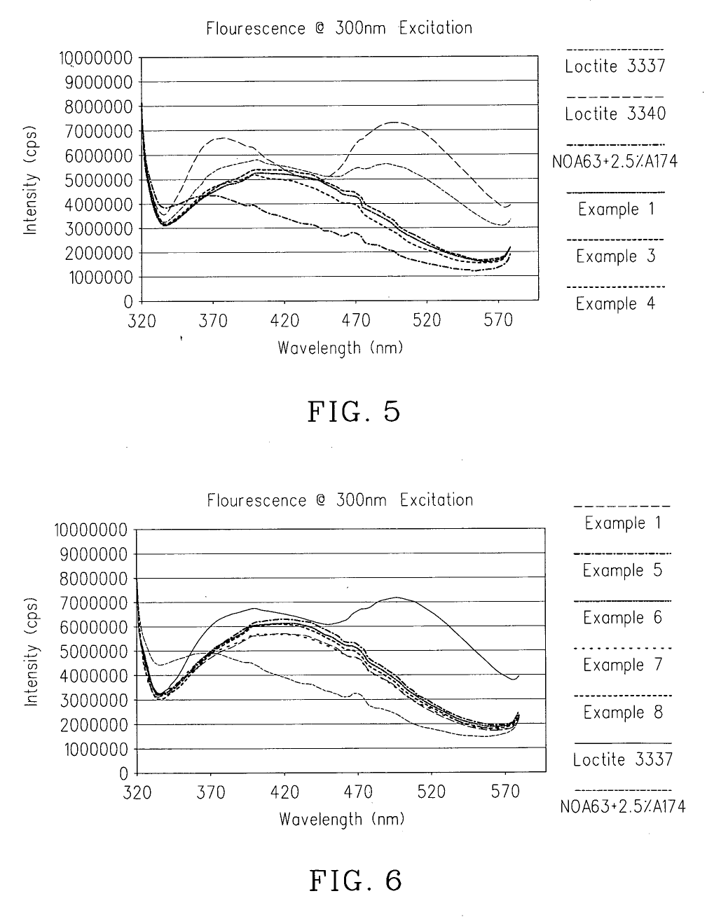

The different compositions of several candidate cationically photocurable adhesives 208 are provided below and then the results of various tests on some of these exemplary compositions are described with respect to FIGS. 5-15.

### EXAMPLE 1

### Composition

| | | |
|---|---|---|
| 35.00% | Stepanpol PD-200LV | Polyol |
| 32.00% | Cyracure UVR-6128 | Epoxy |
| 2.50% | Silquest A-186 | Epoxy Functional Silane |
| 0.50% | Rhordorsil 2074 | Photoinitiator |
| 1.00% | Esacure KIP 150 | Photosensitizer |
| 29.00% | DEN 438 | Epoxy |

### EXAMPLE 2

### Composition

| | | |
|---|---|---|
| 74.00% | Nanopox XP21/0316 | Silica Filled Epoxy |
| 20.00% | Poly [di (ethylene | Polyol |
| | glycol) phthalate] diol | |
| | MW = 576 | |
| 2.50% | Silquest A-186 | As Above |
| 2.50% | Rhordorsil PC-702 | Photoinitiator |
| 1.00% | Esacure KIP 150 | As Above |

### EXAMPLE 3

### Composition

| | | |
|---|---|---|
| 32.00% | Epon Resin 160 | Epoxy |
| 29.00% | Cyracure 6110 | Epoxy |
| 35.00% | Stepanpol PD-200LV | As Above |
| 2.50% | Silquest A-186 | As Above |
| 0.50% | Rhodorsil 2074 | As Above |
| 1.00% | Esacure KIP 150 | As Above |

### EXAMPLE 4

### Composition

| | | |
|---|---|---|
| 25.00% | Epon 1001F | Epoxy |
| 41.00% | Cyracure UVR-6110 | As Above |
| 30.00% | Stepanpol PD-200LV | As Above |
| 2.50% | Silquest A-186 | As Above |
| 0.50% | Rhodorsil 2074 | As Above |
| 1.00% | Esacure KIP 150 | As Above |

### EXAMPLE 5

### Composition

Same composition as in Example 1 but substitute Sarcat CD-1012 [4-[(2-hydroxytetradecyl)oxy]phenyl]phenyliodonium hexafluoro antimonate (Sartomer Co. Exton, Pa.) for the Rhodorsil 2074. Photoinitiator.

### EXAMPLE 6

### Composition

Same composition as in Example 1 but substitute General Electric UV9392C (Phenyl-4-octyloxyphenyl iodonium hexaflouro antimonate) (GE Silicones, Waterford, N.Y.) for the Rhodorsil 2074. Photoinitiator.

### EXAMPLE 7

### Composition

Same composition as in Example 1 but substitute Irgacure 250, (a 75% solution of (4-methylphenyl)-4-(2-methylpropyl) phenyl iodonium hexaflouro phosphate in propylene carbonate) (Ciba Geigy Corp., Tarrytown, N.Y.) for the Rhordorsil 2074. Photoinitiator

### EXAMPLE 8

### Composition

| | | |
|---|---|---|
| 35.00% | Stepanpol PD-110LV | Polyol |
| 33.00% | Cyracure UVR-6110 | As Above |
| 28.00% | DEN 438 | As Above |
| 2.50% | Silquest A-186 | As Above |
| 0.50% | Rhodorsil 2074 | As Above |
| 1.00% | Esacure KIP150 | As Above |

### EXAMPLE 9

### Composition

| | | |
|---|---|---|
| 29.00% | Cyracure UVR-6110 | As Above |
| 32.75% | Cyracure UVR-6128 | As Above |
| 35.00% | Stepanpol PD-200LV | As Above |
| 2.50% | Silquest A-186 | As Above |
| 0.50% | Rhodorsil 2074 | As Above |
| 0.25% | Esacure KIP150 | As Above |

### EXAMPLE 10

### Composition

| | | |
|---|---|---|
| 64.50% | Nanopox XP22/0314 | Silica Filled Epoxy |
| 32.25% | K-Flex 188 | Polyol |
| 2.50% | Silquest A-186 | As Above |
| 0.50% | Rhodorsil 2074 | As Above |
| 0.25% | Esacure KIP150 | As Above |

### EXAMPLE 11

### Composition

Same composition as in Example 10 but substitute K Flex 148 for the K Flex 188.

### EXAMPLE 12

### Composition

| | | |
|---|---|---|
| 43.00% | Nanopox XP22/0314 | As Above |
| 21.50% | Polyset PC-2003 | Epoxy |
| 32.25% | K Flex 188 | As Above |
| 2.50% | Silquest A-186 | As Above |
| 0.50% | Rhodorsil 2074 | As Above |
| 0.25% | Esacure KIP150 | As Above |

### EXAMPLE 13

### Composition

| | | |
|---|---|---|
| 43.27% | Nanopox XP22/0314 | As Above |
| 21.63% | Polyset PC-2003 | As Above |
| 32.25% | K Flex 188 | As Above |
| 2.50% | Silquest A-186 | As Above |
| 0.25% | Rhodorsil 2074 | As Above |
| 0.10% | Esacure KIP150 | As Above |

### EXAMPLE 14

### Composition

| | | |
|---|---|---|
| 47.15% | Cyracure UVR-6128 | As Above |
| 25.00% | Polyset PC-2000 | Cycloaliphatic epoxy |
| | | functional silicone |
| | | oligomer (Polyset Corp., |
| | | Mechanicville, NY) |
| 25.00% | K-Flex 148 | As Above |

| | | |
|---|---|---|
| 2.50% | Silquest A-186 | As Above |
| 0.25% | Rhodorsil 2074 | As Above |
| 0.10% | Esacure KIP150 | As Above |

The Example Adhesives #s 1-14 in addition to Loctite 3337, Loctite 3340, Norland 63+2%% Silquest A-174 all underwent a fluorescence analysis. To prepare for the fluorescence analysis, the liquid adhesive compositions were drawn down onto 2″×3″ glass microscope slides using a 6 mil Bird applicator. The samples were then UV cured by passing under a Fusion Systems 300 W/in D type lamp at 8.5 ft/min and a UV dose of ˜2J/cm2. After this, the samples were left to set under ambient laboratory conditions for at least 24 hours before performing the fluorescence measurements. The fluorescence measurements were taken on a Flourolog-3 Jobin Yvon Spex Horiba Model FL3-21 Fluorimeter for FIGS. 5-7, and a Hitachi Model F-2000 Fluorescence Spectrophotometer for FIGS. 8-11.

Referring to FIG. 5, there is a graph that illustrates the fluorescence curves at an 300 nm excitation wavelength for the Loctite 3337 adhesive, Loctite 3340 adhesive, Norland NOA63+2%% Silquest A-174 adhesive and Example Adhesive #s 1, 3 and 4. As can be seen, the Loctite 3337 and 3340 adhesives all passed the testing but had an unacceptable amount of fluorescence. Norland NOA63+2%% Silquest A-174 had an acceptable fluorescence curve. Although this adhesive had a very low fluorescence it also had inconsistent bonding and cycotoxicity results. It should also be noticed that the fluorescence measured for the Example Adhesive #s 1, 3 and 4 are significantly lower than for the Loctite adhesives.

Referring to FIG. 6, there is a graph that illustrates the fluorescence curves at an 300 nm excitation wavelength for the Loctite 3337 adhesive, Norland NOA63+2%% Silquest A-174 adhesive and Example Adhesive #s 1, 5, 6, 7 and 8. This graph shows that the alternate iodonium cationic photoinitiators in Example Adhesive #s 5, 6, and 7 as well as the different epoxy resins in Example Adhesive # 8 have little effect on the fluorescence of Example Adhesive #1 and all have less fluorescence than Loctite 3337 at the 300 nm excitation wavelength.

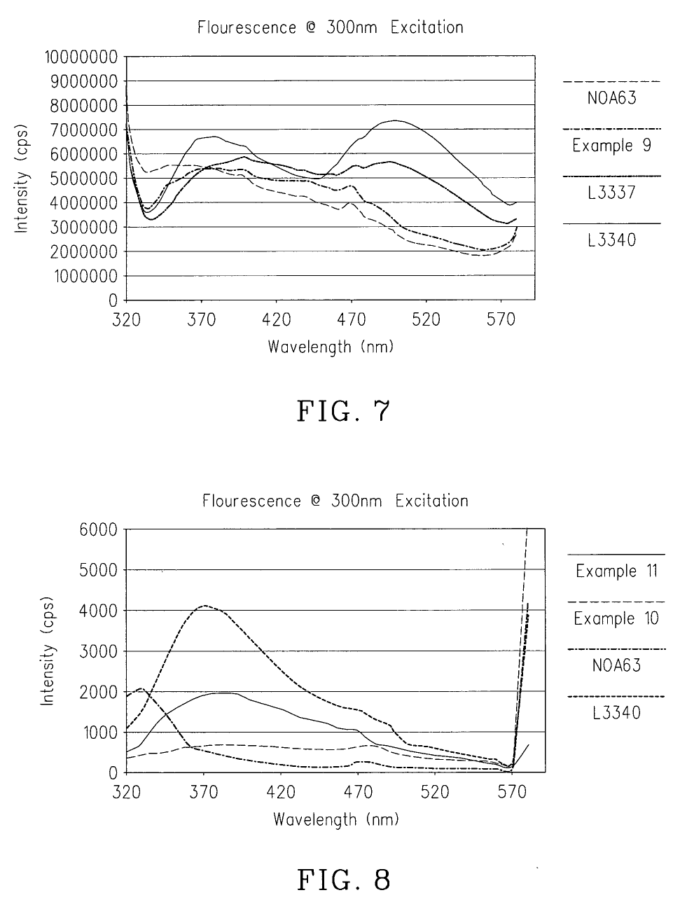

Referring to FIG. 7, there is a graph that illustrates the fluorescence curves at an 300 nm excitation wavelength for the Loctite 3337 adhesive, Loctite 3340 adhesive, Norland NOA63 adhesive and Example Adhesive #9. This graph shows that the Example Adhesive # 9 had less fluorescence at the 300 nm excitation wavelength than Loctite 3337 and 3340 and had fluorescence that was closer to the low fluorescing Norland NOA 63.

Referring to FIG. 8, there is a graph that illustrates the fluorescence curves at an 300 nm excitation wavelength for the Loctite 3340 adhesive, Norland NOA63 adhesive and Example Adhesive #s 10 and 11. This graph shows that Example Adhesive #s 10 and 11 had a lower fluorescence than Loctite 3340 at the 300 nm excitation wavelength.

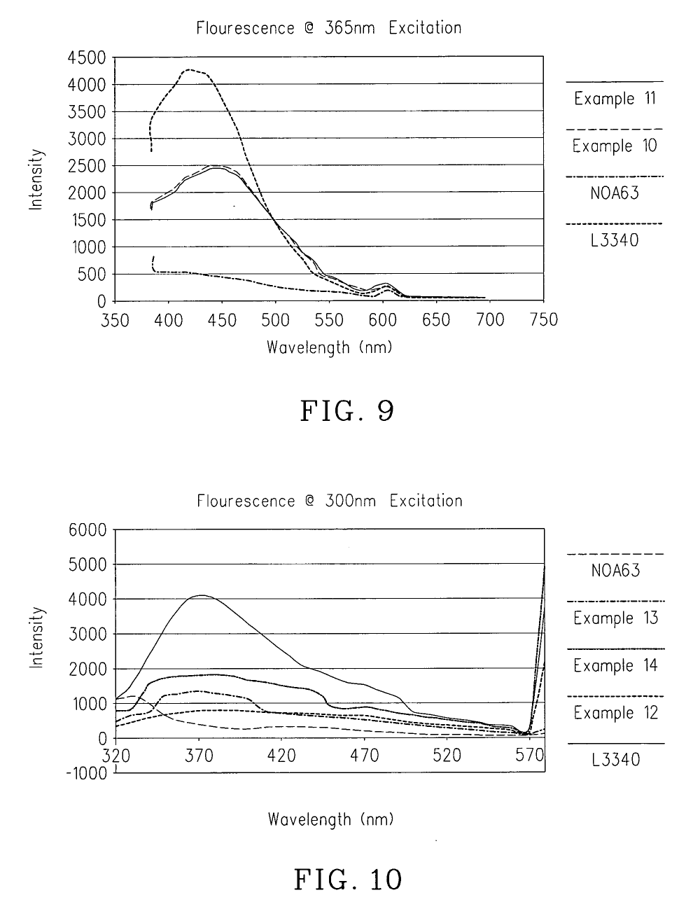

Referring to FIG. 9, there is a graph that illustrates the fluorescence curves at an 365 nm excitation wavelength for the Loctite 3340 adhesive, Norland NOA63 adhesive and Example Adhesive #s 10 and 11. This graph shows that Example Adhesive #s 10 and 11 had a lower fluorescence than Loctite 3340 at the 365 nm excitation wavelength.

Referring to FIG. 10, there is a graph that illustrates the fluorescence curves at an 300 nm excitation wavelength for the Loctite 3340 adhesive, Norland NOA63 adhesive and Example Adhesive #s 12, 13 and 14. This graph shows that Example Adhesive #s 12, 13 and 14 had a lower fluorescence than Loctite 3340 at the 300 nm excitation wavelength. And, the fluorescence for Example Adhesive #s 12 and 13 were close to Norland NOA63.

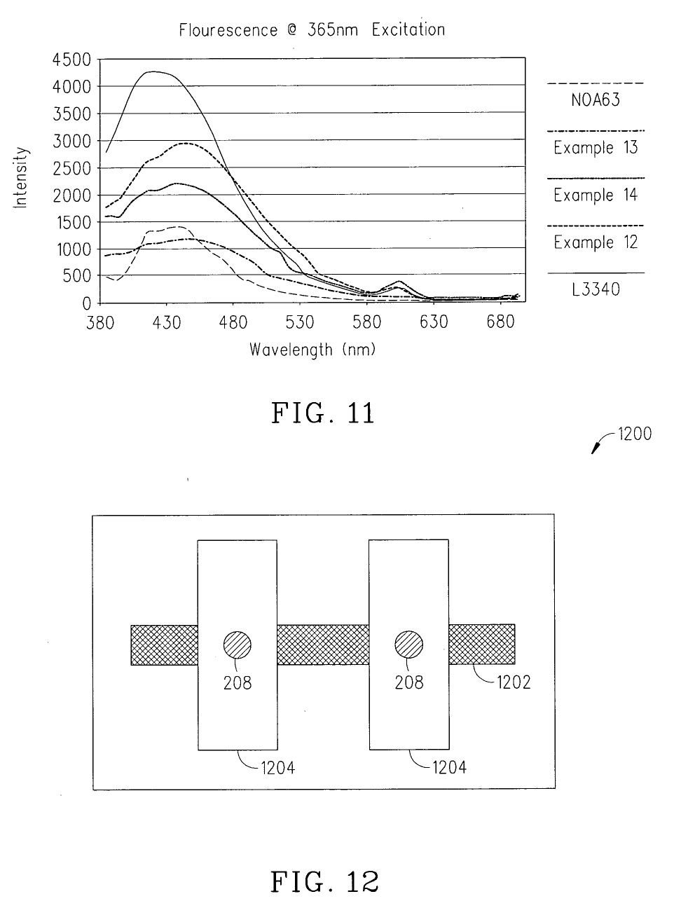

Referring to FIG. 11, there is a graph that illustrates the fluorescence curves at an 365 nm excitation wavelength for the Loctite 3340 adhesive, Norland NOA63 adhesive and Example Adhesive #s 12, 13 and 14. This graph shows that Example Adhesive #s 12, 13 and 14 had a lower fluorescence than Loctite 3340 at the 365 nm excitation wavelength. And, the fluorescence for Example Adhesive # 13 was close to Norland NOA63.

In addition to the fluorescence tests, samples of Example Adhesive #s 1-14, Loctite 3337, Loctite 3340 and Norland 63+2½%% Silquest A-174 were prepared for a tensile adhesion test. The purpose of the tensile adhesion test was to measure the adhesion of these candidate adhesives to glass microscope slides and polystyrene plastic after being immersed in 50° C. water for 72 hours. The test was intended to be used to predict the adhesive performance when bonding glass bottoms onto polystyrene microplate bodies.

To perform the test, black, high impact polystyrene (Fina PS-625) test bars (½″×5″×⅛″) were made using an injection mold. Immediately before bonding, the polystyrene bars were treated for five minutes in a UV/ozone treater (UVO Cleaner Model 342) with an oxygen purge. The polystyrene bars were at 5 mm distance from the UV light. No cleaning/pretreatment was done on glass microscope slides (Fisherfinest Premium Microscope Slides).

To keep specimen alignment and bond spacing uniform, a bonding fixture 1200 was machined out of aluminum (see FIG. 12). First, the polystyrene test bar 1202 was placed in the deepest slot in the bonding fixture 1200. And, then 5 μL of the candidate adhesive 280 was applied at two locations on the polystyrene test bar 1202. Two microscope slides 1204 were then laid over the two adhesives 208 and the polystyrene test bar 1202. The adhesives 208 were cured with one pass at high speed (belt speed 23 ft/min, ˜1000 mJ/cm2) under a Fusion D-bulb (Fusion Systems Model #LC6 benchtop conveyor system. The bonding fixture 1200 was designed to bond two microscope slides 1204 using only one polystyrene test bar 1202. After the adhesives 208 cured, the polystyrene test bar 1202 was cut producing two test specimens. The specimens were placed in a 50% relative humidity/room temperature chamber overnight. Then the specimens were immersed into 50° C. water for 72 hours.

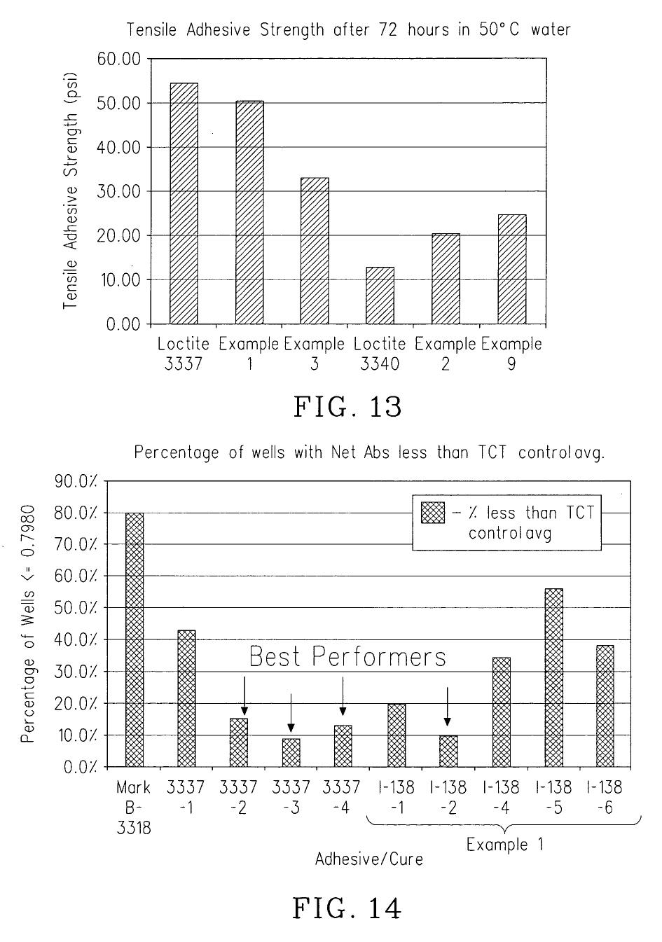

After soaking for 72 hours in 50° C. water, the specimens were tested immediately. Excess water was blotted off the specimens, and they were loaded into a tensile testing fixture mounted in an Instron Model 4202 Tensile Tester. The tensile testing fixture then pulled apart each specimen in tensile mode. And, the load (lbf) was recorded as a function of the linear displacement (in) of the tensile testing fixture. The area of the bond was measured and the maximum load obtained before the bond was broken and then this measurement was then converted to bond strength (psi). Ten specimens were bonded for several candidate adhesives 208 and the graph in FIG. 13 depicts the average bond strength for these adhesives. It should be appreciated that the bonding force for Loctite 3340 and Example Adhesive #s 1, 2, 3 and 9 was deemed acceptable (see FIG. 13).

Referring to FIG. 14, there is a graph that illustrates the results from a cytotoxity test for the Loctite 3337 adhesive and Example Adhesive # 1. As can be seen, the Example Adhesive #1 performed in a comparable manner to the Loctite 3337.

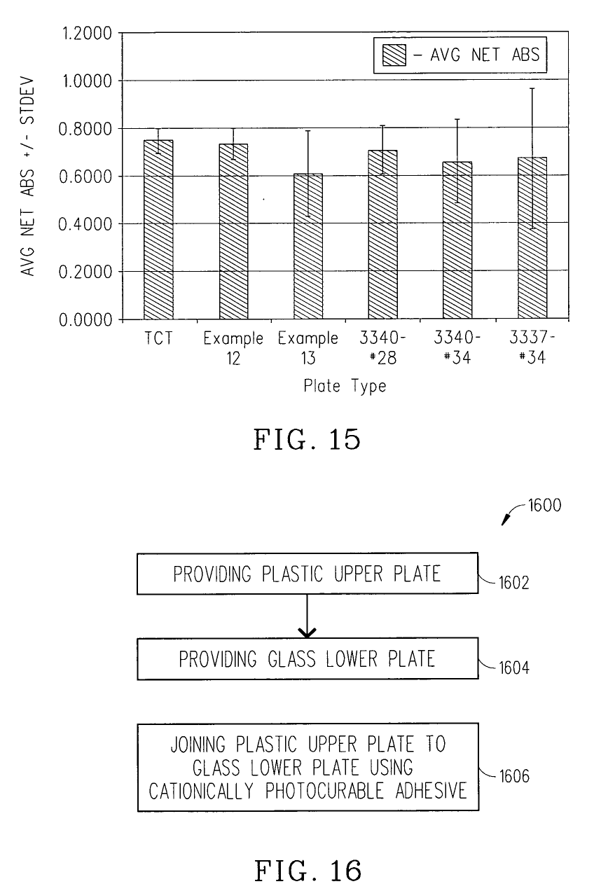

Referring to FIG. 15, there is a graph that illustrates the results from a cytotoxity test for the Loctite 3337 adhesive, Loctite 3340 adhesive and Example Adhesive #s 12 and 13. As can be seen, the Example Adhesive #s 12 and 13 performed in a comparable manner to Loctite 3340 and 3337.

Referring to FIG. 16, there is a flowchart illustrating the steps of the preferred method 1600 for making the multiwell plate 100. Although the multiwell plate 100 is described herein as having ninety-six functional wells arranged in a grid having a plurality of rows and columns, again it should be understood that the present invention is not limited to any specific number of wells. Accordingly, the multiwell plate 100 and preferred method 1600 should not be construed in such a limited manner.

The multiwell plate 100 can be manufactured by providing (step 1602) an upper plate 200 and also providing (step 1604) a lower plate 220. The upper plate 200 has a frame that forms the sidewalls 240 of one or more wells 160 and is preferably made from a polymeric material such as polystyrene. And, the lower plate 220 has a layer that forms the bottom walls 260 of the wells 160 and is preferably made from a glass plate. The next process step in manufacturing the multiwell plate 100 includes joining (step 1606) the upper plate 200 to the lower plate 220 using a low fluorescence, low cytotoxicity cationically photocured adhesive 280. In the preferred embodiment, the joining step 1606 includes: (1) applying a substantially thin film of the adhesive 280 onto one of the plates 200 or 220; (2) placing the other plate 220 or 200 onto the plate 200 or 220 that had the adhesive 280 applied thereto; and (3) directing a UV light to initiate the cure of the adhesive 280. Again, the cationically photocured adhesive 280 includes: (1) one or more photocationally polymerizable epoxy and/or oxetane functional resins; (2) a low fluorescing cationic photoinitiator; and (3) a low fluorescing photosensitizer if the cationic photoinitiator does not have an adequate absorption at a wavelength >280 nm to initiate cure.

Following are some additional features, advantages and uses of the multiwell plate 100, method 1600 and the cationically photocured adhesive 280 of the present invention:
The preferred adhesives 280 used to assemble glass bottom multiwell plates 100 and other multiwell products ideally have no odor, no volatile components, no extractables, be non-autofluorescent, and non-cytotoxic. The preferred adhesives 280 should also be able stand up to liquid submersion without delamination, sometimes for extended periods of time.
Competitive products on the market today cannot grow cells due to contamination from their adhesives. The preferred adhesives 280 described herein minimizes the contamination of the working surfaces of a multiwell plate by providing a bottom surface that cells grow very well on.
The preferred cationically cured adhesives 208 are not inhibited by atmospheric oxygen which leads to well cured surfaces. This is because the adhesives 208 undergo “living” polymerizations such that they continue to cure once the polymerization process has started. This “dark cure” continues after the part's exposure to uv light has ceased, allowing the adhesive's cure to continue until high percentages of the initial monomer are covalently incorporated into the polymerized adhesive. The resulting low level of contamination also provides for usable GAPS coatings in plate bottoms.
Glass bottom multiwell plates require the bonding of a thin flat glass piece to a multi-well plastic manifold, where these materials differ greatly in their surface activity and coefficient of thermal expansion. The large differences in materials properties makes the selection or development of adhesive chemistry critical for reliable product performance. The adhesives 280 of the present invention meets these needs.
It should be appreciated that the cationic adhesives 280 can be formulated to have very low outgassing (<0.01% for Loctite 3338 for example) and low shrinkage (3%) where low shrinkage is important for reducing interfacial stress upon cure which can lead to delamination).
It should also be appreciated that the cationic adhesives 280 can be formulated from many different types of epoxy monomers that have no odor, low cytotoxicity, and have room temperature vapor pressures of <0.01 mm Hg (several listed as Nil such as Cyracure UVR-6110 and 6128 for example). In addition, the photoinitiators for UV curing these adhesives 280 should have vapor pressures of <0.03 mm Hg (UVI-6976 and 6992) and are used at only 1% of the formulation weight or so. The formulations of the adhesives 280 can be made to have a lower modulus by the inclusion of polyol crosslinkers that also have vapor pressures of <0.01 mm Hg (Tone 0301, 0305 and 0310 for example). Moreover, IPNs can be formed by the inclusion of vinyl ethers in the formulation of the adhesive 280 that provide very low modulus to the formulation (<1000 psi which leads to very low interfacial stress for the cured part). In addtion, polydimethylsiloxanes and polybutadienes with pendant epoxide groups may be used for cured formulations with low modulus. Because these flexible epoxies are non-hygroscopic, they allow for less sensitivity to relative humidity's effect on cure rate and bestow physical properties of the cured adhesive 280 that vary little with water submersion. Elastomer particles such as EPDM, PDMS, CTBN and Viton powders can also be added to reduce modulus (example: Santoprene) and shrinkage. Low outgassing epoxy silanes are also available to enhance adhesion to various substrates including glass and various metal oxides.
It should be appreciated that the preferred adhesive need not be a cationically photocurable adhesive so long as it has some of the aforementioned physical properties such as low cytotoxicity, a tensile adhesion that is >0.10 MPa, a substrate curl that is >70 mm and/or a low outgassing in the range of <0.01% (for example).

Although several embodiments of the present invention has been illustrated in the accompanying Drawings and described in the foregoing Detailed Description, it should be understood that the invention is not limited to the embodiments disclosed, but is capable of numerous rearrangements, modifications and substitutions without departing from the spirit of the invention as set forth and defined by the following claims.

1. A multiwell plate comprising:
an upper plate that forms sidewalls of at least one well, the upper plate being formed from a polymeric material; and
a lower plate that forms a bottom wall of the at least one well, the lower plate being formed from glass, wherein said upper plate and said lower plate are attached and bound to one another by a cationically photocured adhesive.

2. The multiwell plate of claim 1, wherein said adhesive includes:
an epoxy and/or oxetane functional material; and
a photoinitiator (<1.0 wt %).

3. The multiwell plate of claim 2, wherein said adhesive further includes a photosensitizer (<1.0 wt %).

4. The multiwell plate of claim 2, wherein said adhesive further includes a polyol which has a level where a ratio of epoxy equivalents to hydroxyl equivalents is >1.5.

5. The multiwell plate of claim 2, wherein said adhesive includes an epoxy functional silane (<10.0 wt %).

6. The multiwell plate of claim 2, wherein said adhesive includes elastomer particles.

7. The multiwell plate of claim 2, wherein said epoxy functional group has a terminal, pendant, internal or cyclic ring.

8. The multiwell plate of claim 2, wherein said epoxy functional material is a cycloaliphatic epoxy functional material.

9. The multiwell plate of claim 2, wherein said epoxy functional material is a non-hygroscopic epoxy functional material.

10. The multiwell plate of claim 2, wherein said photoinitiator is a low fluorescence photoinitiator.

11. The multiwell plate of claim 2, wherein said photoinitiator is an iodonium salt.

12. The multiwell plate of claim 1, wherein said adhesive has a Young's modulus that is >20 MPa.

13. The multiwell plate of claim 1, wherein said adhesive has a glass transition temperature that is >25° C.

14. The multiwell plate of claim 1, wherein said adhesive has a tensile adhesion that is >0.10 MPa.

15. The multiwell plate of claim 1, wherein said adhesive has a substrate curl that is >70 mm.

16. The multiwell plate of claim 1, wherein said adhesive has a low fluorescence at excitation wavelengths between 300-550 nm.

17. The multiwell plate of claim 1, wherein said adhesive has a low cytotoxicity.

18. The multiwell plate of claim 1, wherein said adhesive has low outgassing in the range of <0.01%.

19. A method for making a multiwell plate, said method comprising the steps of:
providing an upper plate that forms sidewalls of at least one well, said upper plate made from a polymeric material;
providing a lower plate that forms a bottom wall of the at least one well, said lower plate made from glass; and
joining said upper plate to said lower plate using a cationically photocurable adhesive.

20. The method of claim 19, wherein said step of joining further includes the steps of:
applying a substantially thin film of the cationically photocurable adhesive onto one of said plates;
placing said other plate onto the one plate; and
directing a ultraviolet light to initiate the cure of the cationically photocurable adhesive.

21. The method of claim 19, wherein said adhesive includes:
an epoxy and/or oxetane functional material; and
a photoinitiator (<1.0 wt %).

22. The method of claim 21, wherein said adhesive further includes a photosensitizer (<1.0 wt %).

23. The method of claim 21, wherein said adhesive further includes a polyol which has a level where a ratio of epoxy equivalents to hydroxyl equivalents is >1.5.

24. The method of claim 21, wherein said adhesive includes an epoxy functional silane (<10.0 wt %).

25. The method of claim 21, wherein said adhesive includes elastomer particles.

26. The method of claim 21, wherein said epoxy functional group is a terminal, pendant, internal or cyclic ring.

27. The method of claim 21, wherein said epoxy functional material is a cycloaliphatic epoxy functional material.

28. The method of claim 21, wherein said epoxy functional material is a non-hygroscopic epoxy functional material.

29. The method of claim 21, wherein said photoinitiator is a low fluorescence photoinitiator.

30. The method of claim 21, wherein said photoinitiator is an iodonium salt.

31. The method of claim 19, wherein said adhesive has a Young's modulus that is >20 MPa.

32. The method of claim 19, wherein said adhesive has a glass transition temperature that is >25° C.

33. The method of claim 19, wherein said adhesive has a tensile adhesion that is >0.10 MPa.

34. The method of claim 19, wherein said adhesive has a substrate curl that is >70 mm.

35. The method of claim 19, wherein said adhesive has a low fluorescence at excitation wavelengths between 300-550 nm.

36. The method of claim 19, wherein said adhesive has a low cytotoxicity.

37. The method of claim 19, wherein said adhesive has low outgassing in the range of <0.01%.

38. A multiwell plate used to perform cell-based assays, said multiwell plate comprising:
a frame that forms sidewalls of at least one well, the frame being formed from a polymeric material; and
a layer that forms a bottom wall of the at least one well, the layer being formed from a glass plate, wherein said frame and said layer are attached and bound to one another by a cationically photocured adhesive that includes:
an epoxy and/or oxetane functional material;
a photoinitiator (<1.0 wt %); and
a photosensitizer (<1.0 wt %) if the photoinitiator does not have an adequate absorption at a wavelength >280 nm to initiate cure.

39. The multiwell plate of claim 38, wherein said adhesive further includes a polyol which has a level where a ratio of epoxy equivalents to hydroxyl equivalents is >1.5.

40. The multiwell plate of claim 38, wherein said adhesive further includes an epoxy functional silane (<10.0 wt %).

41. The multiwell plate of claim 38, wherein said adhesive includes elastomer particles.

42. The multiwell plate of claim 38, wherein said photoinitiator is a low fluorescence photoinitiator such as an iodonium salt.

43. A multiwell plate used to perform cell-based assays, said multiwell plate comprising:
a frame that forms sidewalls of at least one well, the frame being formed from a polymeric material; and
a layer that forms a bottom wall of the at least one well, the layer being formed from a glass plate, wherein said frame and said layer are attached and bound to one another by a low cytotoxicity adhesive that has the following properties:
a tensile adhesion that is >0.10 MPa;
a substrate curl that is >70 mm; and
a low outgassing in the range of <0.01%.

44. The multiwell plate of claim 43, wherein said adhesive has a low fluorescence at excitation wavelengths between 300-550 nm.

45. The multiwell plate of claim 43, wherein said adhesive has a Young's modulus that is >20 MPa.

46. The multiwell plate of claim 43, wherein said adhesive has a glass transition temperature that is >25° C.

47. The multiwell plate of claim 43, wherein said adhesive has low extractables.
